(19) United States
(12) Patent Application Publication
(10) Pub. No.: US 2025/0073141 A1
(43) Pub. Date: Mar. 6, 2025

(54) SILICONE-FREE COSMETIQUE COMPOSITIONS

(71) Applicant: L'OREAL, Paris (FR)

(72) Inventor: Nicholas STEBBINS, Clark, NJ (US)

(73) Assignee: L'OREAL, Paris (FR)

(21) Appl. No.: 18/238,678

(22) Filed: Aug. 28, 2023

(51) Int. Cl. A61K 8/31; A61Q 19/00

(52) U.S. Cl. CPC A61K 8/31; A61Q 19/00; A61K 2800/34

(57) ABSTRACT

The instant disclosure relates to compositions including squalene at least one bio-based branched hydrocarbon), at least one bio-based pentaerythrityl ester as well as to methods of using such compositions. The cosmetic composition is free of silicone. The cosmetic composition is easily spreadable and has a favorable sensory without the use of silicones.

### FIELD OF THE INVENTION

The instant disclosure is generally directed to a cosmetic composition having and methods of using cosmetic composition. More specifically, the instant disclosure is directed to compositions containing an emollient blend that contributes to sensory texture comparable to compositions texture containing silicones.

### BACKGROUND OF THE INVENTION

A variety of compositions, especially cosmetic compositions, have been developed to provide easy and comfortable application onto a targeted substrate. These compositions include one or more active ingredients that have demonstrated biological effectiveness against skin aging.

However, in addition to the benefits of these products on the skin, consumers are also looking for a pleasant sensoriality at the time of application, and/or after application. As a result, these products traditionally contain silicone compounds. Silicones are well known in cosmetics products to provide a smooth application with minimal drag, a soft end-feel, minimal greasiness, and no tack. All these characteristics are desired in products.

However, the formulation of environmentally friendly cosmetic products, i.e. whose design and development take into account environmental issues, is becoming a major concern to help meet global challenges.

It is therefore essential to propose compositions and/or preparation processes and/or more sustainable ingredients to meet these environmental challenges.

In this context, it is important to develop new cosmetic compositions with a better carbon footprint, in particular by promoting the use of renewable raw materials and/or with a good index of naturalness and/or natural origin and more particularly of plant origin while reducing the use of compounds of petrochemical origin.

There is therefore in particular a need for new care compositions having a sensoriality comparable to existing care compositions, while being devoid of silicone compounds.

There is in particular a need for care compositions devoid of silicone compounds having a sensoriality comparable to the existing care compositions at the time of application, as well as a pleasant sensation after application.

### BRIEF SUMMARY OF THE INVENTION

The summary is provided to introduce a selection of concepts in a simplified form that are further described below in the detailed description of the invention. This summary is not intended to identify key features of the claimed subject matter, nor is it intended to be used as an aid in determining the scope of the claimed subject matter.

Indeed, the inventors have discovered surprisingly that the specific association of squalene, bio-based branched hydrocarbon and bio-based pentaerythrityl ester in specific proportions solves the technical problems mentioned above. This combination advantageously allows to remove silicone compounds without deteriorating the sensoriality of the composition, both on application and after application.

These compositions also include sustainable ingredients, thus making it possible to meet environmental challenges.

The cosmetic compositions typically include:
a. Squalene
b. at least one bio-based branched hydrocarbon;
c. at least one bio-based pentaerythrityl ester; and
wherein the cosmetic composition is substantially free of silicone.

Such composition according to the instant disclosure may be in the form of emulsions, dispersions, gels, balm and stick. In some embodiments, the cosmetic compositions is in the form of a gel.

In some embodiments, the squalene is present from about 0.1 to about 20 wt. % based on the total weight of the composition. In some embodiments, the squalene is present from about 0.15 to about 18 wt. % based on the total weight of the composition.

In one or more embodiments, the at least one bio-based branched alkane is selected from the group selected from C9-12 alkane, C15-19 alkane, C15-23 alkane, branches isomers of undecane, branches isomers of tridecane and mixture thereof. In some embodiments, the at least one bio-based branched alkane is present from about 0.1 to about 20 wt. % based on the total weight of the composition. In various embodiments, the at least one bio-based branched alkane is present from about 0.5 to about 18 wt. % based on the weight of the composition.

In some embodiments, the at least one bio-based pentaerythrityl ester is selected from the group consisting of Pentaerythrityl Tetraisostearate; Dipentaerythrityl Hexacaprylate/Hexacaprate; Pentaerythrityl Distearate; Dipentaerythrityl Tetrahydroxystearate/Tetraisostearate; Pentaerythrityl Isostearate/Caprate/Caprylate/Adipate; Pentaerythrityl Tetrabehenate; Pentaerythrityl Tetralaurate; Pentaerythrityl Tetrapelargonate and mixture thereof. In one or more embodiments, the at least one bio-based pentaerythrityl ester is present from about 0.1 to about 20 wt. % based on the total weight of the composition. In various embodiments, the least one bio-based pentaerythrityl ester is present from about 0.15 to about 18 wt. % based on the total weight the composition.

In some embodiments, the cosmetic compositions are free of silicones.

In one or more embodiments, the cosmetic compositions might comprise water.

In one or more embodiments, the cosmetic compositions might comprise one or more additional components selected from the group consisting of actives selected from humectant, antimicrobial, antioxidant, preservative, vitamin, vitamin derivative, UV filter, vegetable extract; and dye/pigment, filler, thickener, polymer, penetrant, fragrance, dispersant, film-forming agent; ceramide; opacifier and combinations thereof.

In various embodiments, the cosmetic compositions might include one or more actives selected from the group consisting of sodium hydroxide, disodium EDTA, sodium citrate, sodium hyaluronate, capryloyl salicylic acid, lactic acid, methyl dihydro jasmonate, acetyl trifluoromethyl phenyl valyglycine, pentaerythrityl tetra-di-t-butyl hydroxydrocinnamate, n-hydroxysuccinimide, palmitoyl oligopeptide, chrysin, palmitoyl tetrapeptide-7, yeast extract, citric acid and combinations thereof.

In some embodiments, the compositions may comprise one or more actives selected from the group consisting of sodium hydroxide, disodium EDTA, sodium citrate, sodium hyaluronate, capryloyl salicylic acid, lactic acid, methyl dihydro jasmonate, acetyl trifluoromethyl phenyl valyglycine, pentaerythrityl tetra-di-t-butyl hydroxydrocinnamate, n-hydroxysuccinimide, palmitoyl oligopeptide, chrysin, palmitoyl tetrapeptide-7, yeast extract, citric acid and combinations thereof.

In accordance with another aspect of the disclosure, the cosmetic composition may include:
a. From about 0.1 to about 20% wt. of Squalene;
b. From about 0.1 to about 20% wt. of at least one bio-based branched hydrocarbon;
c. From about 0.1 to about 20% wt. of at least one bio-based pentaerythrityl ester; and
wherein the cosmetic composition is substantially free of silicone.

The cosmetic compositions are useful for treating skin, for example, the skin of the face and neck of a human. Thus, the instant disclosure relates to methods of treating the skin comprising application of the cosmetic composition of the instant disclosure to the skin. The cosmetic compositions are additionally useful in methods for treating skin dryness, repairing skin damage due to photoaging, and diminishing the appearance of wrinkles, dark spots, and uneven skin texture. The aforementioned methods may be non-therapeutic.

Other features and advantages of the present invention will be apparent from the following more detailed description, taken in conjunction with the accompanying drawings which illustrate, by way of example, the principles of the invention.

This disclosure describes exemplary embodiments in accordance with the general inventive concepts and is not intended to limit the scope of the invention in any way. Indeed, the invention as described in the specification is broader than and unlimited by the exemplary embodiments set forth herein, and the terms used herein have their full ordinary meaning.

### DETAILED DESCRIPTION OF THE INVENTION

“Cosmetically acceptable” means compatible with any keratinous substrate. For example, “cosmetically acceptable carrier” means a carrier that is compatible with any keratinous substrate.

The articles “a” and “an,” as used herein, mean one or more when applied to any feature in embodiments of the present invention described in the specification and claims. The use of “a” and “an” does not limit the meaning to a single feature unless such a limit is specifically stated. The article “the” preceding singular or plural nouns or noun phrases denotes a particular specified feature or particular specified features and may have a singular or plural connotation depending upon the context in which it is used. The adjective “any” means one, some, or all indiscriminately of whatever quantity.

“At least one,” as used herein, means one or more and thus includes individual components as well as mixtures/combinations.

By “substantially free of silicone”, as used herein, means that the composition comprises less than 1% by weight relative to the total weight of the composition, preferably less than 0.5% by weight, preferably less than 0.3% by weight, preferably less than 0.1% by weight, more preferably from 0.01% to 1% by weight of silicone. Preferably, the composition is completely free of silicone. By silicone is meant any silicone compound.

By “bio-based”, as used herein, means that the RM is being comprised of at least 50% by molecular weight from renewable carbons or from recycled feedstocks/byproducts.

The transitional terms “comprising”, “consisting essentially of” and “consisting of”, when used in the appended claims, in original and amended form, define the claim scope with respect to what unrecited additional claim elements or steps, if any, are excluded from the scope of the claim(s). The term “comprising” is intended to be inclusive or open-ended and does not exclude any additional, unrecited element, method, step or material. The term “consisting of” excludes any element, step or material other than those specified in the claim and, in the latter instance, impurities ordinary associated with the specified material(s). The term “consisting essentially of” limits the scope of a claim to the specified elements, steps or material(s) and those that do not materially affect the basic and novel characteristic(s) of the claimed invention. All materials and methods described herein that embody the present invention can, in alternate embodiments, be more specifically defined by any of the transitional terms “comprising,” “consisting essentially of,” and “consisting of.”

Other than in the operating examples, or where otherwise indicated, all numbers expressing quantities of ingredients and/or reaction conditions are to be understood as being modified in all instances by the term “about,” meaning within 10% of the indicated number (e.g. “about 10%” means 9%-11% and “about 2%” means 1.8%-2.2%).

All percentages and ratios are calculated by weight unless otherwise indicated. All percentages are calculated based on the total composition unless otherwise indicated. Generally, unless otherwise expressly stated herein, “weight” or “amount” as used herein with respect to the percent amount of an ingredient refers to the amount of the raw material comprising the ingredient, wherein the raw material may be described herein to comprise less than and up to 100% activity of the ingredient. Therefore, weight percent of an active in a composition is represented as the amount of raw material containing the active that is used, and may or may not reflect the final percentage of the active, wherein the final percentage of the active is dependent on the weight percent of active in the raw material.

All ranges and amounts given herein are intended to include subranges and amounts using any disclosed point as an end point. Thus, a range of “1% to 10%, such as 2% to 8%, such as 3% to 5%,” is intended to encompass ranges of “1% to 8%,” “1% to 5%,” “2% to 10%,” and so on. All numbers, amounts, ranges, etc., are intended to be modified by the term “about,” whether or not so expressly stated. Similarly, a range given of “about 1% to 10%” is intended to have the term “about” modifying both the 1% and the 10% endpoints. Further, it is understood that when an amount of a component is given, it is intended to signify the amount of the active material unless otherwise specifically stated.

Notwithstanding that the numerical ranges and parameters setting forth the broad scope of the disclosure are approximations, unless otherwise indicated the numerical values set forth in the specific examples are reported as precisely as possible. Any numerical value, however, inherently contains certain errors necessarily resulting from the standard deviation found in their respective testing measurements. The example that follows serves to illustrate embodiments of the present disclosure without, however, being limiting in nature.

The instant disclosure relates to cosmetic compositions that contain a specific association of squalene, bio-based branched hydrocarbon and bio-based pentaerythrityl ester in specific proportions. This combination advantageously allows to remove silicone compounds without deteriorating the sensoriality of the composition, both on application and after application.

The cosmetic compositions of the instant disclosure typically include:
a. Squalene
b. at least one bio-based branched hydrocarbon;
c. at least one bio-based pentaerythrityl ester; and
wherein the cosmetic composition is substantially free of silicone.

The cosmetic compositions are generally formulated in the form of emulsions, dispersions, gels. In some embodiments, the cosmetic compositions is in the form of a gel.

As discussed above, it is believed that the specific association of squalene, at least one bio-based branched hydrocarbon and at least one bio-based pentaerythrityl ester exhibits a sensory comparable to the cosmetic compositions that contain silicones.

Suitable components, such as those listed below, may be included or excluded from the formulations for the cosmetic compositions depending on the specific combination of other components, the form of the cosmetic compositions, and/or the use of the formulation (e.g., a lotion, a serum, gel, cream, etc.).

### Squalane

The cosmetic compositions include squalene.

Squalane (C 30 H62, chemical name 2,6,10,15,19,23-hexamethyltetracosane) is a saturated hydrocarbon composed of a chain of 30 saturated carbon atoms with the following formula:

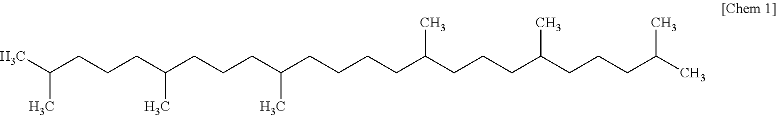 [SMILES: CC(C)CCCC(C)CCCC(C)CCCCC(C)CCCC(C)CCCC(C)C]

It is obtained by hydrogenation of squalene (C₃₀H₅₀). Originally, squalene was extracted from shark liver oil. Squalane, due to its saturated chemical structure, is much more stable than squalene which peroxides very easily.

The squalane used in the invention is preferably of plant origin. Preferably, squalane comes from vegetable oils, preferably olive oil, wheat germ oil, bran oil, rice oil or olive starches.

Preferably, in the present invention, squalane is extracted from olive oil.

For example, the product marketed under the name Neossance Squalane by Amyris will be used.

Squalene is typically present in the cosmetic composition in an amount of from about 0.1 wt. % to about 20 wt. %, based on the total weight of the cosmetic composition. For example, the cosmetic compositions may include an amount of squalene ranging from 0.1 wt. % to about 20 wt. %, about 0.15 wt. % to about 20 wt. %, about 0.2 wt. % to about 20 wt. %, about 0.25 wt. % to about 20 wt. %, about 0.3 wt. % to about 19 wt. %, about 0.4 wt. % to about 19 wt. %, about 0.5 wt. % to about 18 wt. %, or about 1 wt. % to about 15 wt. %, including ranges and sub-ranges there between, based on the total weight of the cosmetic composition. Additionally or alternatively, the amount of squalene may be 0.1 wt. % to 20 wt. %, 0.2 wt. % to 20 wt. %, 0.3 wt. % to 20 wt. %, 0.35 wt. % to 20 wt. %, 0.35 wt. % to 15 wt. %, 0.5 wt. % to 14 wt. %, 0.6 wt. % to 14 wt. %, or 0.8 wt. % to 12 wt. % including ranges and sub-ranges there between, based on the total weight of the composition. Likewise, in some embodiments, the cosmetic composition may include an amount of squalene that is about 20 wt. %, about 19 wt. %, about 18 wt. %, about 17 wt. %, about 16 wt. %, about 15 wt. %, about 14 wt. %, about 13 wt. %, about 12 wt. %, about 11 wt. %, about 10 wt. %, about 9 wt. %, about 8 wt. %, about 7 wt. %, about 6 wt. %, about 5 wt. %, about 4 wt. %, about 3 wt. %, about 2 wt. %, about 1 wt. %, about 0.5 wt. %, or about 0.1 wt. %, based on the total weight of the cosmetic composition. In one embodiment, the amount of squalene may be 2% or about 2% of the total weight of the cosmetic composition.

### Bio-Based Branched Hydrocarbon

The cosmetic composition according to the instant disclosure may comprise a mixture of branched alkanes having from 9 to 19 carbon atoms.

As an example of alkanes suitable for the invention, mention may be made of mixtures of alkanes having from 15 to 19 carbon atoms having a molecular weight between 200 g/mol and 250 g/mol.

Preferably, the alkanes are branched.

Preferably, alkanes are of plant origin.

Preferably, a mixture comprising 95 to 99% by weight relative to the total weight of the mixture, branched alkanes C 15-C 19, and 1 to 5% by weight relative to the total weight of the mixture, alkanes C 12-C 14 and C 20-C 26. Such a mixture preferably has a molecular weight of about 216 g/mol. Such a mixture is marketed by Seppic under the reference Emogreen L15.

Alternatively, preferably, a mixture comprising 95 to 99% by weight relative to the total weight of the mixture, branched alkanes C 15-C 19, and 1 to 5% by weight relative to the total weight of the mixture, alkanes C 12-C 14 and C 20-C 26. Such a mixture preferably has a molecular weight of about 248 g/mol. Such a mixture is marketed by Seppic under the reference Emogreen L19.

Alternatively, preferably, a mixture comprising 10% by weight relative to the total weight of the mixture, branched alkanes C 15-C 19, and 1 to 5% by weight relative to the total weight of the mixture, alkanes C 12-C 14 and C 20-C 26. Such a mixture is marketed by Seppic under the reference Emosmart L19.

The cosmetic composition includes bio-based branched hydrocarbon typically in an amount from about 0.1 wt. % to about 20 wt. %, based on the total weight of the cosmetic composition. In some instances, the amount of bio-based branched hydrocarbon present in the cosmetic composition may be from about 0.1 wt. % to about 20 wt. %, about 0.15 wt. % to about 20 wt. %, about 0.2 wt. % to about 20 wt. %, about 0.25 wt. % to about 20 wt. %, about 0.3 wt. % to about 19 wt. %, about 0.4 wt. % to about 19 wt. %, about 0.5 wt. % to about 18 wt. %, or about 1 wt. % to about 15 wt. %, including ranges and sub-ranges there between, based on the total weight of the cosmetic composition.

In one embodiment, the amount of bio-based branched hydrocarbon may be 1% or about 1% of the total weight of the cosmetic composition.

### Bio-Based Pentaerythrityl Ester

The cosmetic composition according to the instant disclosure may comprise at least one or more bio-based pentaerythrityl ester.

Esters of pentaerythritol and mono or fatty diacids, C6-C22, such as the mixture of pentaerythritol ester and isostearic, capric, caprylic and adipic acids (Croda Supermol-L).

Polyesters comprising at least three ester functions, mono- or poly-carboxylic acids, saturated, unsaturated or aromatic, linear, branched or cyclic, optionally hydroxylated, C 4-C 40 and respectively polyols or monoalcohols C 2-C 40, preferably C 3-C 40; said polyester optionally comprising at least one free hydroxyl.

For example, mention may be made of oils comprising three ester functions, acid comprising three carboxylic functions, monohydroxylated, and monoalcohol C 2-C 4.

For example, mention may be made of linear fatty acid esters having a total carbon number ranging from 35 to 70 such as pentaerythrityl tetrapelargonate (MW=697 g/mol).

Also suitable are fatty alcohol esters or branched fatty acids, such as pentaerythrityl tetraisononanoate (MW=697 g/mol), pentaerythrityl tetraisostearate (MW=1202 g/mol), polyglyceryl tetraisostearate-2 (MW=1232 g/mol), and also those described in EP-A-0 955 039, such as tetradecyl-2-tetradecanoate pentaerythrityl (MW=1538 g/mol).

As an example of bio-based pentaerythrityl ester suitable for the invention, mention may be made of Pentaerythrityl Tetraisostearate; Dipentaerythrityl Hexacaprylate/Hexacaprate Pentaerythrityl Distearate; Dipentaerythrityl Tetrahydroxystearate/Tetraisostearate; Pentaerythrityl Isostearate/Caprate/Caprylate/Adipate; Pentaerythrityl Tetrabehenate; Pentaerythrityl Tetralaurate; Pentaerythrityl Tetrapelargonate and mixture thereof.

Bio-based pentaerythrityl ester that may be mentioned include Pentaerythrityl Tetraisostearate, such as the commercial product sold under the trade name CRODAMOL PTIS-LQ-(MV) by Croda or JOLEE 7181 by Oleon or PAESTER PETIS and PAESTER PETIS/MB by Patech Fine Chemicals; Dipentaerythrityl Hexacaprylate/Hexacaprate, such as the commercial product sold under the trade name DUB DPHCC or DUB DPHCC MB by Stearinerie Dubois, Pentaerythrityl Distearate, such as the commercial product sold under the trade name CUTINA PES by BASF, Dipentaerythrityl Tetrahydroxystearate/Tetraisostearate, such as the commercial product sold under the trade name COSMOL 168 EV or SALACOS 168 EV by Nisshin Oillio; Pentaerythrityl Isostearate/Caprate/Caprylate/Adipate, such as the commercial product sold under the trade name SP SUPERMOL L MBAL-LQ-(RB) or SUPERMOL L-LQ-(RB) by Croda.

In some embodiments, the at least one bio-based pentaerythrityl ester is present from about 0.1 to about 20 wt. % based on the total weight of the cosmetic composition. In one or more embodiments, the at least one bio-based pentaerythrityl ester is present from about 0.15 to about 18 wt. % based on the total weight of the cosmetic composition.

The cosmetic composition includes bio-based bio-based pentaerythrityl ester typically in an amount from about 0.1 wt. % to about 20 wt. %, based on the total weight of the cosmetic composition. In some instances, the amount of bio-based bio-based pentaerythrityl ester present in the cosmetic composition may be from about 0.1 wt. % to about 20 wt. %, about 0.15 wt. % to about 20 wt. %, about 0.2 wt. % to about 20 wt. %, about 0.25 wt. % to about 20 wt. %, about 0.3 wt. % to about 19 wt. %, about 0.4 wt. % to about 19 wt. %, about 0.5 wt. % to about 18 wt. %, or about 1 wt. % to about 15 wt. %, including ranges and sub-ranges there between, based on the total weight of the cosmetic composition.

In one embodiment, the amount of bio-based bio-based pentaerythrityl ester may be 1% or about 1% of the total weight of the cosmetic composition.

The cosmetic composition includes bio-based branched hydrocarbon typically in an amount from about 0.1 wt. % to about 20 wt. %, based on the total weight of the cosmetic composition. In some instances, the amount of bio-based branched hydrocarbon present in the cosmetic composition may be from about 0.1 wt. % to about 20 wt. %, about 0.15 wt. % to about 20 wt. %, about 0.2 wt. % to about 20 wt. %, about 0.25 wt. % to about 20 wt. %, about 0.3 wt. % to about 19 wt. %, about 0.4 wt. % to about 19 wt. %, about 0.5 wt. % to about 18 wt. %, or about 1 wt. % to about 15 wt. %, including ranges and sub-ranges there between, based on the total weight of the cosmetic composition.

In one embodiment, the amount of bio-based branched hydrocarbon may be 0.8% or about 0.8% of the total weight of the cosmetic composition.

### Cosmetically Acceptable Carrier System

The cosmetic compositions include a cosmetically acceptable carrier system. The term “cosmetically acceptable” means a carrier that is compatible with any keratinous substrate, and for purposes hereof, includes water and optionally water based solvents subject to any exclusions as disclosed herein.

The cosmetic compositions may comprise any constituent normally employed in the topical application and administration envisaged. Mention may in particular be made of water, solvents, fatty compounds (i.e. described by the International Federation Societies of Cosmetic Chemists, for example, in Cosmetic Raw Material Analysis and Quality, Volume I: Hydrocarbons, Glycerides, Waxes and Other Esters (Redwood Books, 1994), which is incorporated herein by reference in its entirety), polyols, pigments, fillers, silicones, surfactants, thickeners, gelling agents, preservatives and their mixtures in all proportions.

### Organic Solvent

In accordance with the disclosure, one or more organic solvent is present in the composition. The organic solvent present in the cosmetic composition, according to the disclosure, includes, but is not limited to, butylene glycol, C12-15 alkyl benzoate, glycerin, propylene, glycol, caprylyl glycol, or combinations thereof. Although these solvents are given as an example, it will be appreciated that other solvents compatible with cosmetic applications known in the art may be used.

In accordance with the various embodiments, organic solvent may be present in a given composition in an amount of from about 10% to about 30%, alternatively from about 12% to about 28%, alternatively from about 15% to about 25%, or any suitable combination, sub-combination, range, or sub-range thereof by weight, based on the weight of the composition. One of ordinary skill in the art, however, will appreciate that other ranges are within the scope of the invention

### Water

In accordance with the various embodiments, water may be present in a given composition in an amount of from about 2% to about 90%, about 3% to about 90%, about 5% to about 85%, from about 10% to about 80%, alternatively from about 15% to about 75%, alternatively from about 20% to about 70%, alternatively from about 30% to about 65% or any suitable combination, sub-combination, range, or sub-range thereof by weight, based on the weight of the composition. One of ordinary skill in the art, however, will appreciate that other ranges are within the scope of the invention.

### Polymeric Thickener

In accordance with the various embodiments, the composition may include one or more polymeric thickeners. In some embodiments, the one or more thickener may be selected from one or more of natural gums and synthetic polymers, for example, the thickener may be selected from the group consisting of starches (corn, rice, tapioca, potato), gums (xanthan carrageenan, gellan, sclerotium, tarabiotech fermentation). In some particular embodiments, the thickener may be selected from acrylates/C10-30 alkyl acrylate crosspolymer, carbomer, xanthan gum, hydroxypropyl guar, Ceratonia siliqua (carob) gum, ammonium polyacryloyldimethyl taurate, ammonium acryloyldimethyltaurate/steareth-25 methacrylate crosspolymer, and polyacrylate crosspolymer-6.

In some embodiments, the polymeric thickener may be one of carbomer, acrylates/C10-30 alkyl acrylate crosspolymer, polyacrylate crosspolymer-6, microcrystalline cellulose (and) cellulose gum, xanthan gum, sodium carboxymethyl starch, sclerotium gum (and) xanthan gum, xanthan gum (and) Ceratonia siliqua (carob) gum (50/50), dehydroxanthan gum, hydroxypropyl starch phosphate, sclerotium gum (and) xanthan gum (75/25), sclerotium gum, xanthan gum (and) sclerotium gum (and) Lecithin (and) pullulan, or combinations thereof.

In some embodiments the composition may comprise two or more polymeric thickeners. The amount of each of the at least one polymeric thickener, when present, may be present in the cosmetic composition in a range of from about 0.01% to about 2%, or from about 0.01% to about 1.5%, or from about 0.3% to about 1.2%, or any suitable combination, sub-combination, range, or sub-range thereof by weight, based on the weight of the composition. One of ordinary skill in the art, however, will appreciate that other ranges are within the scope of the invention.

In some embodiments, the total amount of polymeric thickener in the cosmetic composition, when present, is present from about 0.01% to about 5%, or from about 0.02% to about 2%, or from about 0.03% to about 1.5%, or from about 0.1% to about 0.2%, or any suitable combination, sub-combination, range, or sub-range thereof by weight, based on the weight of the composition. One of ordinary skill in the art, however, will appreciate that other ranges are within the scope of the invention.

Thus, one or more polymeric thickener, when present, is present by weight, based on the total weight of the composition, from about 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.5, 4.0, 4.5 to about 5.0 percent, including increments and ranges therein and there between.

### Optional Active Compounds

In accordance with the disclosure, embodiments of the cosmetic composition may include one or more additional compounds selected from active compounds, in particular keratinous tissue actives, such as, but not limited to, skin actives. In some embodiments, optional active compounds may be selected from the group consisting of: vitamins, for example, Vitamin E, carnosine, hyaluronic acid, panthenol, at least one sun filter, at least one hydroxy acid, and combinations thereof. In some embodiments, an active compound is selected from the group consisting of alpha, beta or polyhydroxy acids selected from the group consisting of lactic acid, glycolic acid, salicylic acid, malic acid, tartaric acid, citric acid, mandelic acid, lactobionic acid, gluconolactone, galactose, and combinations thereof. For the avoidance of doubt, the cosmetic composition may include one or more of any one of the optional active compounds. For example, the cosmetic composition may include one, or more than one hydroxy acid, one, or more than one vitamin, and the like.

Vitamin E may be selected from the group consisting of alpha-tocopherol, beta-tocopherol, delta-tocopherol, gamma-tocopherol, and alpha-tocotrienol, beta-tocotrienol, delta-tocotrienol, gamma-tocotrienol, and derivatives thereof. Salts or derivatives of tocopherols include pharmaceutically acceptable compounds such as acetate, sulfate, succinate, nicotinate, palmitate, allophanate, phosphate, quinone, or halogenated derivatives, esters, or stereoisomers, for example. The invention encompasses the use of Vitamin E derivatives in which substitutions, additions, and other alterations have been made in the 6-chromanol ring and/or side chain, with the proviso that the derivatives maintain the antioxidant activity of Vitamin E. Additional tocopherols can be constructed by conjugation to the ring structure or side chain of various other moieties, such as those containing oxygen, nitrogen, sulfur and/or phosphorus. Tocopherol derivatives can also be made by modifying the length of the side chain from that found in tocopherols such as alpha-, beta-, delta- and gamma-tocopherol. Tocopherols can also vary in stereochemistry and saturation of bonds in the ring structure and side chain. Additional tocopherol derivatives, including prodrugs, can be made by conjugation of sugars or other moieties to the side chain or ring structure. Tocopherols include without limitation stereoisomers (e.g., + and − stereoisomers of alpha-tocopherol; (+/−) indicates a racemic mixture) or mixtures of structurally distinct tocopherols (e.g., alpha-plus gamma-tocopherol). Tocopherols may be obtained from Roche, Nutley, N.J., for example.

In some embodiments, additional actives can also include at least one hydroxy acid selected from alpha, beta or polyhydroxy acids. Thus, in various embodiments, a hydroxy acid may be selected from the group consisting of lactic acid, glycolic acid, salicylic acid, malic acid, tartaric acid, citric acid, mandelic acid, lactobionic acid, gluconolactone, galactose, and combinations thereof.

In some embodiments, additional actives can also include one or more of the antioxidants selected from the group consisting of mangiferin, baicalin, resveratrol, tannic acid, polyphenols, amino acids and derivatives thereof, imidazoles, carnosine derivatives, carotenoids, carotenes (such as a-carotene, P-carotene, and lycopene), vitamin A, co-enzyme Q10, bioflavonoids, glutathione, plant extracts (such as rosemary extract, olive leaf extracts), green tea extracts, and combinations thereof.

In some particular embodiments, the cosmetic composition includes a combination of active compounds present in the cosmetic composition comprising a hydroxy acid, tocopherol, panthenol, hyaluronic acid, or a combination thereof.

Generally, each of the optional active compounds is present in the cosmetic composition in an amount from about 0.1% to about 30%, and in some embodiments, from about 0.5% to about 30%, and in some embodiments, from about 0.5% to about 15%, and in some embodiments, from about 0.1% to about 1%, and in some embodiments, from about 1% to about 2%, or any suitable combination, sub-combination, range, or sub-range thereof by weight, based on the weight of the cosmetic composition.

Vitamin E and its derivatives, including, but not limited to tocopherol, may be present in the cosmetic composition in an amount from about 0.5% to about 2%, and in some embodiments, from about 0.5% to about 1%, and in some embodiments, from about 1% to about 2.0%, or any suitable combination, sub-combination, range, or sub-range thereof by weight, based on the weight of the cosmetic composition.

One or more of hyaluronic acid, panthenol, and carnosine may be present in the cosmetic composition in an amount from about 0.1% to about 5%, and in some embodiments, from about 0.1% to about 3%, and in some embodiments, from about 0.5% to about 1.0%, or any suitable combination, sub-combination, range, or sub-range thereof by weight, based on the weight of the cosmetic composition.

Hyaluronic acid may be present in the cosmetic composition in an amount from about 0.01% to about 1%, and in some embodiments, from about 0.01% to about 0.1%, and in some embodiments, from about 0.05% to about 0.07%, or any suitable combination, sub-combination, range, or sub-range thereof by weight, based on the weight of the cosmetic composition.

Panthenol may be present in the cosmetic composition in an amount from about 0.1% to about 1%, and in some embodiments, from about 0.1% to about 0.5%, and in some embodiments, from about 0.2% to about 0.4%, and in some embodiments, from about 0.2% to about 0.25%, or any suitable combination, sub-combination, range, or sub-range thereof by weight, based on the weight of the cosmetic composition.

A hydroxy acid may be present in the cosmetic composition in an amount from about 0.25% to about 10%, and in some embodiments, from about 0.5% to about 8%, and in some embodiments, from about 1% to about 5%, and in some embodiments, from about 0.25% to about 0.5%, or any suitable combination, sub-combination, range, or sub-range thereof by weight, based on the weight of the cosmetic composition.

Carnosine may be present in the cosmetic composition in an amount from about 0.10% to about 1%, and in some embodiments, from about 0.20% to about 0.5%, and in some embodiments, from about 0.20% to about 0.25%, or any suitable combination, sub-combination, range, or sub-range thereof by weight, based on the weight of the cosmetic composition.

Thus, in various embodiments, optional active compounds, if present, are present in a cosmetic composition according to the disclosure, and each of the individual components in the ranges as described herein above, from about 0.01, 0.02, 0.03, 0.04, 0.05, 0.06, 0.07, 0.08, 0.09, 0.10, 0.20, 0.30, 0.40, 0.50, 0.60, 0.70, 0.80, 0.90, 1.0, 2, 3, 4, 5, 6, 7, 8, 9, 10, 11, 12, 13, 14, 15, 16, 17, 18, 19, 20, 21, 22, 23, 24, 25, 26, 27, 28 to about 30 percent by weight, including increments and ranges there between.

### Optional Additives

The compositions can also comprise at least one additive used in the cosmetics field which does not affect the properties of the compositions according to the invention, such as, emulsifiers, emollients, fragrances, pH adjusters (citric acid, sodium chloride; neutralizing or pH-adjusting agents (e.g., triethanolamine (TEA) and sodium hydroxide), and combinations thereof), other cosmetically acceptable additives, such as but not limited to, pearlescent agents, silica, and coloring materials; essential oils; fruit extracts, for example, Pyrus Malus (Apple) Fruit Extract, and Aloe Barbadensis Leaf Juice Powder. Although the optional additives are given as examples, it will be appreciated that other optional components compatible with cosmetic applications known in the art may be used.

In accordance with the various embodiments, the amount of one or more additives, alone or in combination, present in the cosmetic composition can be present in the cosmetic composition according to the disclosure in a range from about 0.001% to about 20%, by weight, or from about 0.005% to about 0.01%, or from about 0.01% to about 0.1%, or from about 0.15% to about 5%, or from about 0.40% to about 4%, or from about 0.5% to about 2.5% by weight, or from about 1% to about 2%, or any suitable combination, sub-combination, range, or sub-range thereof by weight, based on the total weight of the composition.

Thus, any one or a combination of additives may be present, by weight, based on the total weight of the composition, each one or the combination present from about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.10, 0.20, 0.30, 0.40, 0.50, 0.60, 0.70, 0.80, 0.90, 1.0, 2, 3, 4, 5, 6, 7, 8, 9, 10, 11, 12, 13, 14, 15, 16, 17, 18, 19 to about 20 percent, by weight, based on the weight of the cosmetic composition, including increments and ranges therein and there between.

Although the optional additives are given as examples, it will be appreciated that other optional components compatible with cosmetic applications known in the art may be used that are suitable. It will be appreciated by a skilled artisan that any optional additives are present only to the extent and in amounts that do not materially adversely affect the basic and novel characteristic(s) of the claimed disclosure. Thus, in some embodiments that include optional additives, such optional additives will not materially adversely affect the solubility of the skin actives of the cosmetic composition. And in some embodiments that include optional additives, such optional additives will not materially adversely affect the cosmetic composition forming a single phase solution.

In some particular embodiments, the cosmetic composition may include optional additives, for example one or more of phenoxyethanol, terephthalylidene dicamphor sulfonic acid, trisodium ethylenediamine disuccinate, sodium citrate, sodium chloride, hydroxyacetophenone sodium benzoate, potassium sorbate, citric acid, caprylyl glycol, trisodium ethylenediamine disuccinate, or combinations thereof. In some embodiments, phenoxyethanol is present as a preservative in the cosmetic composition in a range from about 0.5% to about 2%, and in some embodiments in a range from about 1% to about 2%.

In accordance with the various embodiments, the amount of one or more actives and additives, alone or in combination, when present in the cosmetic composition according to the disclosure can be present in a range from about 0.001% to about 20%, and in some embodiments, from about 0.05% to about 0.01%, and in some embodiments, from about 0.01% to about 0.1%, and in some embodiments, from about 0.15% to about 5%, and in some embodiments, from about 0.40% to about 4%, and in some embodiments, from about 0.5% to about 2.5%, and in some embodiments, from about 0.1% to about 0.5%, and in some embodiments, from about 1% to about 2%, or any suitable combination, sub-combination, range, or sub-range thereof by weight, based on the total weight of the cosmetic composition. One of ordinary skill in the art, however, will appreciate that other ranges are within the scope of the invention.

Thus, any one or a combination of actives and additives may be present, each one or the combination present from about 0.001, 0.005, 0.01, 0.02, 0.03, 0.04, 0.05, 0.06, 0.07, 0.08, 0.09, 0.01, 0.02, 0.03, 0.04, 0.05, 0.06, 0.07, 0.08, 0.09, 0.10, 0.20, 0.30, 0.40, 0.50, 0.60, 0.70, 0.80, 0.90, 1.0, 2,3, 4,5, 6,7, 8, 9,10, 11,12, 13, 14,15, 16, 17, 18, 19 to about 20 percent, by weight, including increments and ranges therein and there between.

The following examples are intended to further illustrate the present invention. They are not intended to limit the invention in any way. Unless otherwise indicated, all parts are by weight.

### EXAMPLES

### Example 1—Preparation of Sample Composition

A sample composition set forth in the table below can be prepared as follows:

TABLE 1

| | | Inventive | Comparative |
|---|---|---|---|
| FUNCTION | INCI NAME | Example | Example |
| Fatty Compound | SQUALANE | 2.2 | |
| Bio-Based Branched | C15-19 ALKANE | 1 | |
| Hydrocarbondrocarbon | | | |
| Bio-Based | PENTAERYTHRITYL TETRAISOSTEARATE | 0.8 | |
| Pentaerythrityl Ester | | | |
| | WATER | Q.S. | Q.S. |
| Silicone | CAPRYLYL METHICONE | | 4 |
| Silicone | DIMETHICONE | | 0.2 |
| Polymer | ACRYLATES/C10-30 ALKYL ACRYLATE | 0.15 | 0.3 |
| | CROSSPOLYMER | | |
| Polymer | XANTHAN GUM | 0.35 | 0.3 |
| Surfactant | POLYGLYCERYL-4 CAPRATE; LAURETH-4 | 0.9-1.4 | 0.9-1.4 |
| Preservative | PHENOXYETHANOL | 0.1-0.5 | 0.1-0.5 |
| Chelating Agent | TRISODIUM ETHYLENEDIAMINE | <0.15 | <0.15 |
| | DISUCCINATE | | |
| Solvent | BUTYLENE GLYCOL; ALCOHOL DENAT.; | 2-15 | 4 |
| | PENTYLENE GLYCOL; GLYCERIN | | |
| Skin Active Ingredient | CAFFEINE; CARNOSINE; ARGININE; | 0.5-20 | 0.5-20 |
| | SODIUM THIOSULFATE; CAPRYLIC | | |
| | SALICYLIC ACID; SODIUM | | |
| | HYALURONATE; | | |
| | HYDROXYACETOPHENONE; | | |
| | NIACINAMAIDE | | |
| pH adjuster | CITRIC ACID | <0.6 | <0.5 |

The water, chelating agent(s), preservative(s), and water miscible solvents (minus alcohol) are mixed together in a kettle of sufficient size for the batch. Polymers are then added to the batch with stirring and homogenization to well-disperse the polymers. After time, the batch becomes slightly more viscous and gel-like from the thickening capability of the polymers. Then, surfactants and either silicones or bio-based oils are added to the batch with stirring and homogenization. The batch is left to homogenize until a tight emulsion is formed. Then, water soluble actives are added with stirring and homogenization until all actives are solubilized. Any non-water soluble actives are dissolved in alcohol, if appropriate, and then added to the batch with stirring and homogenization

### Example 2—Testing of Different Emollient Blends

In this section, several emollient blends were tested and compared to one another. The results for each attributes were scored from on scale from 1-5 and tabulated. See Table 2.

TABLE 2
Emollient Blend Comparison Data

| Trial | | Body | Playtime | Greasiness | Cushion | End feel |
|---|---|---|---|---|---|---|
| 1 | Branched alkane (BA) | 1 | 1 | 2 | 1 | 1 |
| 2 | Squalane (S) | 2 | 4 | 5 | 2 | 4 |
| 3 | Pentaerythrityl ester (PE) | 5 | 3 | 3 | 5 | 5 |
| 4 | BA + S | 1 | 2 | 4 | 1 | 2 |
| 5 | BA + PE | | | | | |
| 6 | S + PE | 4 | 4 | 4 | 4 | 4 |
| 7 | BA + S + PE (inventive) | 2 | 5 | 3 | 4 | 4 |
| 8 | Silicone benchmark | 2 | 5 | 2 | 4 | 4 |
| Results | | 1 = light, | 1 = volatile, quick | 1 = minimal | 1 = no | 1 = dry, filmy |
| | | fluid, | absorbing, short | oiliness | cushion | |
| | | flowable | rubbing | | | |
| | | 5 = heavy, | 5 = persistent, | 5 = oily, slick | 5 = high | 5- caring, |
| | | thick | gliding, long | | cushion | silky |
| | | | rubbing | | | |

The examples above demonstrated the difference in sensory and spreadability a blend of emollients could achieve. The goal of the instant disclosure was to develop an emollient blend with favorable sensory and spreadability like that of light and medium silicones in the same base formula.

Since silicone chemistry and their resulting sensory is very unique, it is not obvious how to replace these compounds and there is no clear, universal replacement using carbon-based emollients; thus, a blend was researched and developed.

An emollient blend was developed in a particular base as an example. The base formula was an O/W emulsion serum that contained ACRYLATES/C10-30 ALKYL ACRYLATE CROSSPOLYMER (i.e. Pemulen EZ4U) as the stabilizing polymer, with xanthan gum as thickener to increase viscosity. It also contained various actives, a penetration enhancer, preservatives, solvents, and oil phase. In the comparative case (i.e., Comparative Example 1), the oil phase was comprised of silicone emollients. In the inventive case (i.e., Inventive Example 1), the oil phase was comprised of renewable carbon-based emollients designed to give a sensory similar to Comparative Example that contained the blend of silicones. In selecting the carbon based emollients, a few key sensorial attributes were tested including spreadability during application, lightness/volatility, playtime, cushion, after-feel.

It is well known from the skill in the art that the unique spreadability and lastingness of silicones comes from silicone bonds having a greater bond rotation/flexibility and the raw material itself having a lower surface tension than those found in carbon emollients. Thus, carbon-based emollients with decent spreadability and low stickiness were needed. Without being bound by theory, it is believed that the numerous branch points in molecules can surprisingly increase the spreadability versus the corresponding linear (i.e., unbranched molecule) due to packing and molecular forces enough to make a noticeable difference. Thus, branched carbon-based emollients were selected for study.

Squalane was selected for the overall slip and spreadability, with medium playtime and smoothness without excess oiliness. The molecule has 6 branch points (6 methyl groups at regular intervals) and has an intermediate molecular weight. However, using squalane alone made a formula slightly too oily and does not have much cushion, like it was illustrated Table X, Trial 2.

Branched renewable hydrocarbons (C9-12 alkane, C15-19 alkane) were selected for Trial 1, as they provided quick dissipation while remaining light. However, it was observed that when used alone, the playtime was not nearly long enough, there was minimal cushion, and the end feel is too drying and ‘tight’.

Pentaerythritol is a unique polyol in that is contains 4 —OH groups, each of which can react with a fatty acid to develop a pentaerythrityl ester. Because of the branching contained in these esters, they are high molecular weight, but still liquid and spreadable. For example, pentaerythrityl tetraisostearate contains 4 separate-C:18 chains, each of which has their own branch point. These esters gave a richer feel with great cushion, and increased overall playtime when blended with lighter volatile emollients. If these were used alone, as shown in Trial 3, the overall sensory was too heavy and rich, with poor playtime alone.

However, when the Squalane, the branched hydrocarbon and the pentaterythityl ester were mixed together, as shown in Table 2, Trial 7, the blend demonstrated the balance of important sensorial attributes needed to replace silicones.

Surprisingly, the blend of renewable carbon-based emollients displayed positive sensorial attributes similar to the silicone-containing formula, with low stickiness even in the presence of high levels (>10%) actives and glycerin. Low greasiness was also obtained with the emollient blend, which is another benefit since carbon-based oils can often be perceived to be greasier than silicones.

While the invention has been described with reference to a preferred embodiment, it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment disclosed as the best mode contemplated for carrying out this invention, but that the invention will include all embodiments falling within the scope of the appended claims.

What is claimed is:

1. A cosmetic composition comprising:
a. Squalene
b. at least one bio-based branched hydrocarbon;
c. at least one bio-based pentaerythrityl ester; and
wherein the cosmetic composition is substantially free of silicone.

2. The cosmetic composition of claim 1, wherein the squalene is present from about 0.1 to about 20 wt. % based on the total weight of the composition.

3. The cosmetic composition of claim 1, wherein the at least one bio-based branched alkane is selected from the group selected from C9-12 alkane, C15-19 alkane, C15-23 alkane, branches isomers of undecane, branches isomers of tridecane and mixture thereof.

4. The cosmetic composition of claim 1, wherein the at least one bio-based branched alkane is present from about 0.1 to about 20 wt. % based on the total weight of the composition.

5. The cosmetic composition of claim 4, wherein the at least one bio-based branched alkane is present from about 0.5 to about 18 wt. % based on the total weight of the composition.

6. The cosmetic composition of claim 1, wherein the at least one bio-based pentaerythrityl ester is selected from the group consisting of Pentaerythrityl Tetraisostearate; Dipentaerythrityl Hexacaprylate/Hexacaprate; Pentaerythrityl Distearate; Dipentaerythrityl Tetrahydroxystearate/Tetraisostearate; Pentaerythrityl Isostearate/Caprate/Caprylate/Adipate; Pentaerythrityl Tetrabehenate; Pentaerythrityl Tetralaurate; Pentaerythrityl Tetrapelargonate and mixture thereof.

7. The cosmetic composition of claim 1, wherein the at least one bio-based pentaerythrityl ester is present from about 0.1 to about 20 wt. % based on the total weight of the composition.

8. The cosmetic composition of claim 1, wherein the least one bio-based pentaerythrityl ester is present from about 0.15 to about 18 wt. % based on the total weight the composition.

9. The composition of claim 1, wherein the composition is free of silicones.

10. The composition of claim 1, further comprises water.

11. The composition of claim 10, wherein the water is present from at least 2% based on the total weight the composition.

12. The composition of claim 10, wherein the water is present from about 2% to about 90% based on the total weight the composition.

13. The composition of claim 1, further comprising one or more additional components selected from the group consisting of actives selected from humectant, antimicrobial, antioxidant, preservative, vitamin, vitamin derivative, UV filter, vegetable extract; and dye/pigment, filler, thickener, polymer, penetrant, fragrance, dispersant, film-forming agent; ceramide; opacifier and combinations thereof.

14. The composition of claim 9 further comprising one or more actives selected from the group consisting of sodium hydroxide, disodium EDTA, sodium citrate, sodium hyaluronate, capryloyl salicylic acid, lactic acid, methyl dihydro jasmonate, acetyl trifluoromethyl phenyl valyglycine, pentaerythrityl tetra-di-t-butyl hydroxydrocinnamate, n-hydroxysuccinimide, palmitoyl oligopeptide, chrysin, palmitoyl tetrapeptide-7, yeast extract, citric acid and combinations thereof.

15. A method for treating skin comprising applying a cosmetic composition of claim 1 to the skin.

16. A cosmetic composition comprising:
a. From about 0.1 to about 20% wt. of Squalene;
b. From about 0.1 to about 20% wt. of at least one bio-based branched hydrocarbon;
c. From about 0.1 to about 20% wt. of at least one bio-based pentaerythrityl ester; and
wherein the cosmetic composition is substantially free of silicone; and
wherein all weight percentages are based on the total weight of the cosmetic composition.
